(19) United States
(12) Patent Application Publication
(10) Pub. No.: US 2002/0148467 A1
(43) Pub. Date: Oct. 17, 2002

(54) Emergency breathing apparatus

(76) Inventors: Thomas J. Bosse, Meridian, ID (US); David V. Bosse JR., Winters, CA (US)

Correspondence Address: DYKAS, SHAVER & NIPPER, LLP, P O BOX 877, BOISE, ID, 83701-0877, US

(21) Appl. No.: 09/803,129

(22) Filed: Mar. 8, 2001

(51) Int. Cl. B63C 11/02; A62B 7/00; A61M 15/00; A61M 16/00

(52) U.S. Cl. 128/201.27; 128/202.14

(57) ABSTRACT

A portable emergency breathing apparatus having an inflatable air bladder and a mouth piece. The mouth piece has a valve means for allowing a user to release air stored within the inflatable air bladder to the user's mouth.

### BACKGROUND OF THE INVENTION

1. Field of the Invention

The present invention generally relates to underwater breathing apparatuses, and more particularly relates to underwater emergency apparatuses utilized in an emergency.

2. Background Information

A great need exists for an emergency underwater breathing apparatus for use when a user is trapped under water. Examples of such situations might be in the case of a vehicle which has plunged into a body of water, submarine or other maritime accidents, or in case of a water landing of an aircraft. In such a situation, the amount of time a individual has before his or her air supply runs out is limited. More often than not, if such an individual was able to utilize a source of air while attempting to escape from such an environment, such an individual would be able to survive the catastrophe.

Shown in the prior art are patents which cover emergency breathing apparatuses. U.S. Pat. No. 4,365,628 to Hodel claims an avalanche survival vest. U.S. Pat. No. 4,164,218 to Martin claims a personal escape breathing apparatus. The prior art also shows underwater breathing devices, including U.S. Pat. No. 5,208,226 to Goodley, and U.S. Pat. No. 4,527,657 to Payne.

However, none of these patents disclose a portable emergency breathing apparatus utilizing an inflatable air bladder and a mouth piece having valve means. The present invention solves these needs.

### SUMMARY OF THE INVENTION

The present invention is a portable emergency breathing apparatus. The preferred embodiment of the present invention utilizes an air source in combination with a mouth piece. The preferred air source is an inflatable air bladder, preferably elastic and having at least one handle for allowing a user to grasp the air bladder. This air bladder further has an outlet means for delivering air stored within the bladder to an attached mouthpiece. The air bladder further has at least one air inlet means for allowing the air bladder to be inflated with air. This inlet means, alternatively, could be the mouthpiece itself.

An attached mouthpiece is used for communicating air stored within the air bladder to a user's mouth. The mouthpiece has at least one valve means for allowing a user to selectively release said air from said air bladder through said mouthpiece and into the user's mouth. In a second embodiment the mouthpiece has a second valve means for allowing the user to exhale through the mouthpiece, thereby venting exhausted air either into the air bladder or out a vent.

The present invention can be utilized as an aid to help the user rise to the surface and as a flotation device once the user surfaces. If the present invention is colored a bright color, such as florescent orange, the device could help rescuers spot survivors using the invention. Alternatively, emergency beacons, first aid kits, noseplugs, and the like could be attached to the present invention to make it more useable in an emergency. Another option would be to attach a weight system to the device to allow the user to have a controlled ascent.

Still other objects and advantages of the present invention will become readily apparent to those skilled in this art from the following detailed description wherein I have shown and described only the preferred embodiment of the invention, simply by way of illustration of the best mode contemplated by carrying out my invention. As will be realized, the invention is capable of modification in various obvious respects all without departing from the invention. Accordingly, the drawings and description of the preferred embodiment are to be regarded as illustrative in nature, and not as restrictive.

### BRIEF DESCRIPTION OF THE DRAWINGS

FIG. 1 is a perspective view of one embodiment of the present invention.

FIG. 2 is a perspective view of a second embodiment of the present invention.

FIG. 3 is one embodiment of a mouthpiece or apparatus body for use with the present invention.

FIG. 4 is a cross sectional view of the embodiment shown in FIG. 3.

FIG. 5 shows a second embodiment of the mouthpiece of apparatus body utilized with the present invention.

FIG. 6 shows a third embodiment of the mouthpiece or apparatus body utilized with the present invention.

FIG. 7 shows a cross sectional view of the mouthpiece or apparatus body shown in FIG. 6.

### DESCRIPTION OF THE PREFERRED EMBODIMENTS

While the invention is susceptible of various modifications and alternative constructions, certain illustrated embodiments thereof have been shown in the drawings and will be described below in detail. It should be understood, however, that there is no intention to limit the invention to the specific form disclosed, but, on the contrary, the invention is to cover all modifications, alternative constructions, and equivalents falling within the spirit and scope of the invention as defined in the claims. Also, while this disclosure refers generally to “air,” said terminology is intended to not only include atmospheric air, but any other gas (oxygen, nitrogen, etc.) which a user may choose to utilize with the present invention.

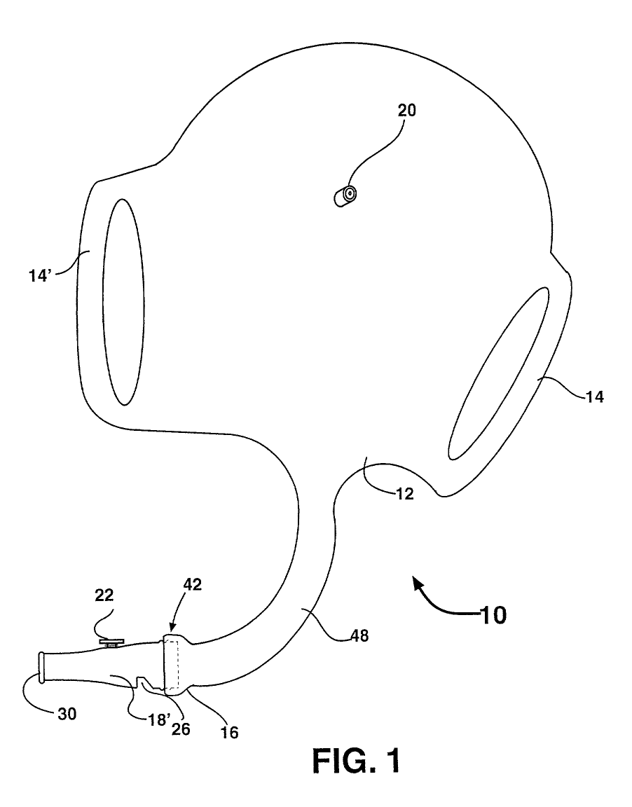

Referring initially to FIG. 1, a first embodiment of the present invention is shown. This figure shows this first embodiment of the invented portable emergency breathing apparatus 10. This embodiment utilizes an inflatable air bladder or other air storage means 12. In the preferred embodiment, this air bladder 12 comprises an elastic rubber bladder. However, other forms of air bladders 12 are also envisioned, including, but not limited to hard cased air bladders, and accordion shaped air bladders.

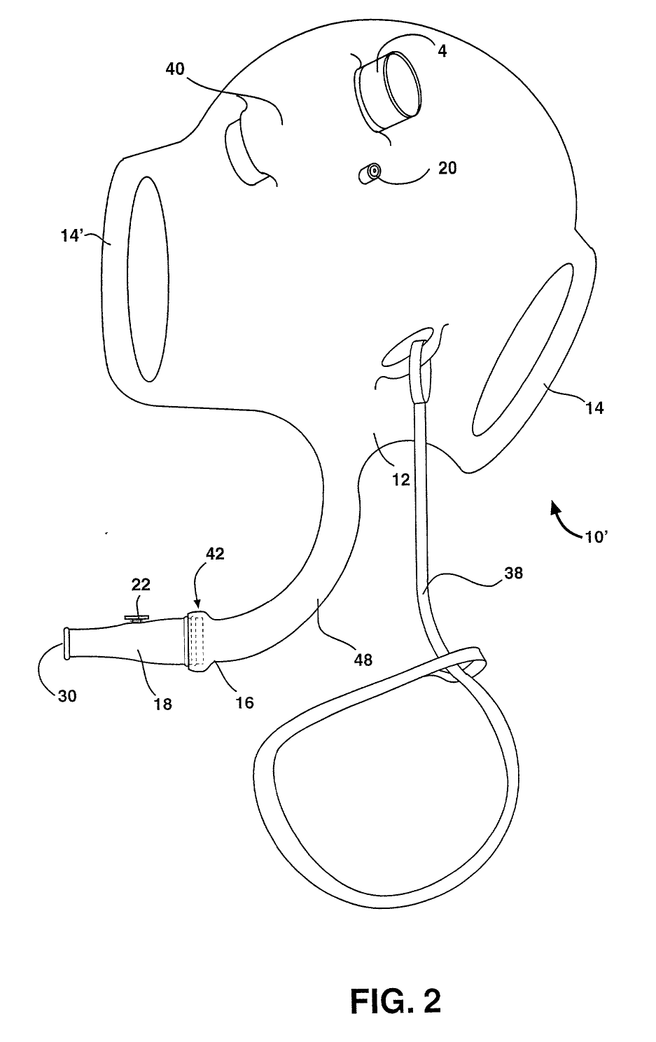

It is preferred that this air bladder 12 further comprise at least one handle 14 for allowing a user to grasp the apparatus 10. More than one handle (14, 14′) may be present to allow for two handed operation, or to allow a second person to hold on to the bladder 12. Additionally, as shown in FIG. 2, a hand strap 38 or other means of attaching the bladder 12 to the user is shown. Other possible features include the use of a connection system for connecting multiple apparatuses together (for instance, in lashing them together on the surface).

Referring again to FIG. 1, the air bladder 12 can either be filled with a supply of air through an inlet means 20, such as the needle connection shown, or through the mouthpiece or apparatus body 18′ which is connected to the outlet means 16 of the air bladder. While these two types of filling means are shown in the figures, it is understood that any method of inflating the air bladder 12 would also be intended to be included within this application. Air supply sources envisioned include, but are not limited to, portable compressed air cylinders, fixed air-line connections, and hand pumps. Storage locations of the apparatuses could either be in individual stored units, or stored in a central location, or even stored in a manner similar to drop down masks similar to the oxygen masks utilized by airlines.

Preferably extending from the air bladder 12 is a neck or umbilical cord 48 extending to the outlet means 16. Inclusion of this neck 48 has many purposes, including the ability for a rescuer to extend an attached mouthpiece 18′ through a small opening (such as a submerged car's window), thereby giving a trapped person access oxygen. Alternatively, this neck 48 could itself include the valve means utilized to release the air supply from the air bladder 12 to the mouthpiece 18′.

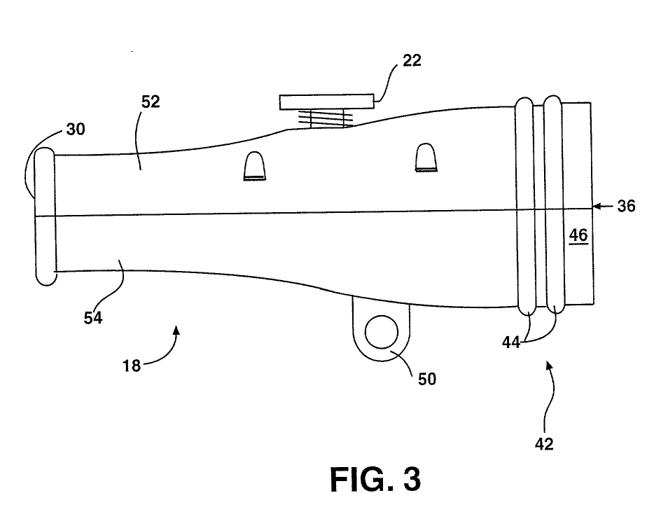

The neck 48 attaches the outlet means 16 to a means for fluidly attaching 42 the air bladder 12 to the mouthpiece 18′. In the FIG. 1 embodiment, the means for fluidly attaching 42 comprises the stretching of the flexible outlet means 16 over the outside surface of the mouthpiece 18 at the mouthpiece's air storage opening end. Such attachment could further be done through use of at least one rib 44 (shown in FIG. 3) located on the outside surface 46 of the mouthpiece 18. Additionally, a ring, band or other fitting could be used to hold the outlet means 16 upon the mouthpiece 18. While this ribbed attachment means is shown in the drawings, it is expressly envisioned that other forms of attachment are also possible.

Referring again to FIG. 1, attaching to the air bladder 12 is the mouthpiece 18′. This mouthpiece 18′ fluidly connects the mouth of a user with the air supply contained within the bladder 12. The mouthpiece 18′ preferably additionally has a manual valve means 22 for allowing the user to selectively release air stored in the air bladder 12 to the mouth of the user. The mouthpiece 18′ has a mouth opening 30 for cooperation with the user's mouth. In the preferred embodiment, the mouthpiece 18′ further comprises a vent channel 26, preferably utilized in conjunction with a second valve means (described later), for allowing the user to exhale back though the mouthpiece 18′. Alternatively, the mouthpiece 18′ could comprise a mask means (not shown) for using the present invention on small children or infants.

Referring now to FIG. 2, a second embodiment of the present invention is shown. In this embodiment, the portable emergency breathing apparatus 10′ has an air bladder or air storage means 12 extending through a neck 48 to a mouthpiece 18. This embodiment further has a flashlight loop 40 for receiving a standard flashlight 4. Additionally, this embodiment utilizes an adjustable hand strap means 38 for allowing the user to secure the apparatus 10′ to his/her wrist.

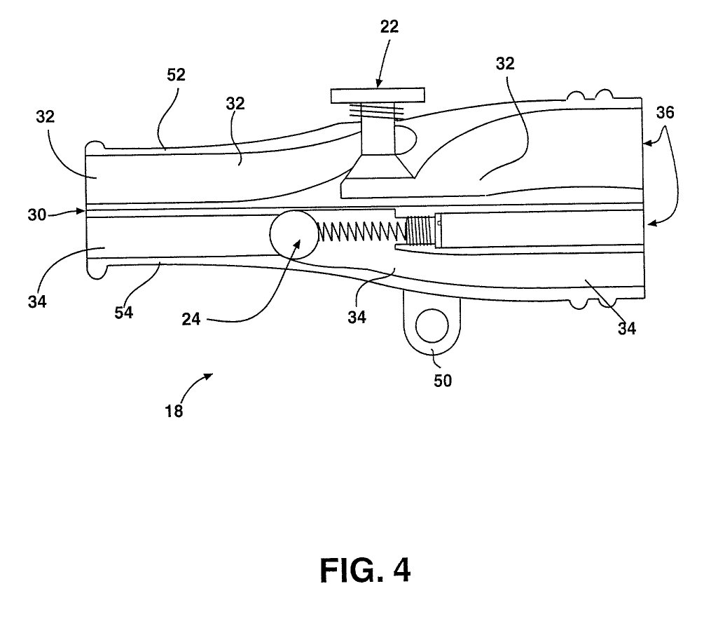

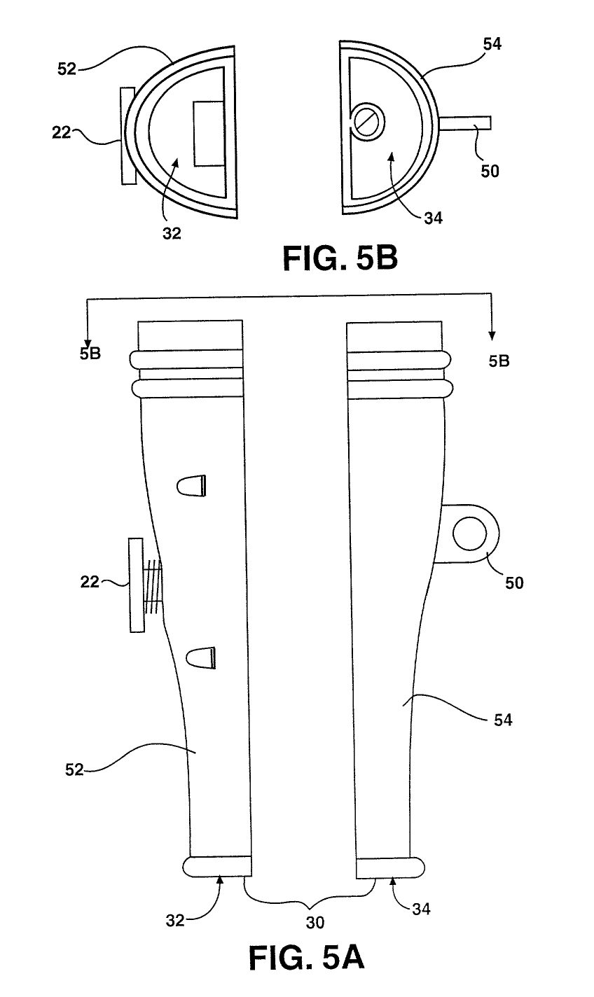

Referring now to FIG. 3, one embodiment of the mouthpiece 18 utilized with the present invention is shown. This embodiment (also shown in FIGS. 4, 5A and 5B) is generally of two-halve construction, having a top half 52 and a bottom half 54. The two halves, in use, are fixed together, preferably either through at least one fastening means.

The mouthpiece 18 has a first end having an air storage opening 36 for fluid connection with the air bladder, and a second end having a mouth opening 30 for use with or in a user's mouth. Attachment of the air bladder to the first end of the mouthpiece 18 can be done through many different attachment means 42. The preferred attachment means 42 of ribs 44 located on the outside surface 46 of the mouthpiece 18 are shown. In such a use, the air bladder, or extension thereof, would be stretched over said outside surface 46, being held on said mouthpiece 18 via the ribs 44 (as generally shown in FIG. 1 and FIG. 2). Alternatively, a clip or other locking device (not shown) could be utilized to hold the bladder on the outside surface 46. The ribs 44 are preferred, but are not necessary, as a proper clip or locking device could lock the bladder on the mouthpiece without use of ribs or other raised features.

Extending from at least one portion of the outside surface 46 of the mouthpiece is a lanyard attachment means 50 for allowing the mouthpiece to be attached to a wrist strap or lanyard (not shown). Also preferably extending from the outside surface 46 of the mouthpiece 18 is at least one manual valve means 22 for allowing a user to release air from the air bladder through the mouthpiece 18 and into the mouth of a user.

Referring now to FIG. 4, a partial cross-sectional view of the embodiment of FIG. 3 is shown. This figure shows the mouthpiece 18 having a pair of air channels or passageways extending therethrough. First, an outlet channel 32 extends from the air storage opening 36 to the mouth opening 30. Interposed in this outlet channel 32 is the first valve means 22. A spring means or other means is utilized to keep this valve 22 closed by default. When the mouthpiece 18 is attached to an air source, such as the air bladder, air pressure is pressurized in the air storage opening 36 end of the outlet channel 32. Upon opening the valve 22, this pressure of air is allowed to flow to the mouth opening 30 end of the mouthpiece and into the mouth of a user. Upon closing the valve 22, this pressure is not longer allowed to flow along that path.

Second, an inlet channel 34 extends from the mouth opening 30 and back through to the air storage opening 36. Interposed in this inlet channel 34 is a second valve means 24. This valve means 24 can be any type of valve, but in the preferred embodiment is a spring activated check valve able be opened through air pressure supplied by the user, either through blowing into the inlet channel 34 via the mouth opening 30 or through other means. In such a manner, a user would be able to inflate the attached air bladder through blowing through the inlet channel 34 or by applying an air source to the inlet channel 34 at the mouth opening 30.

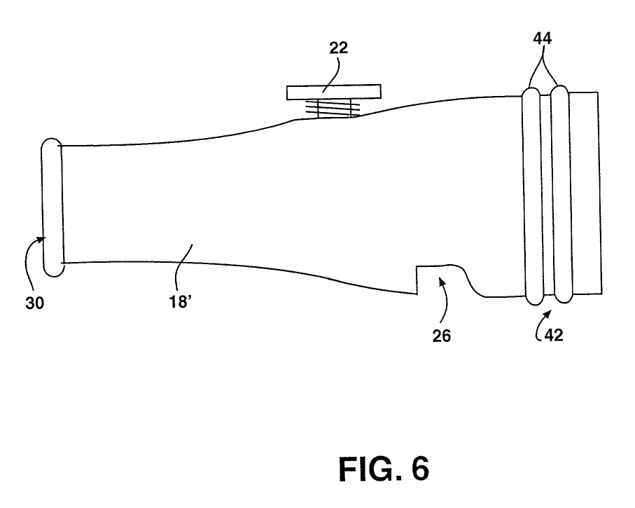

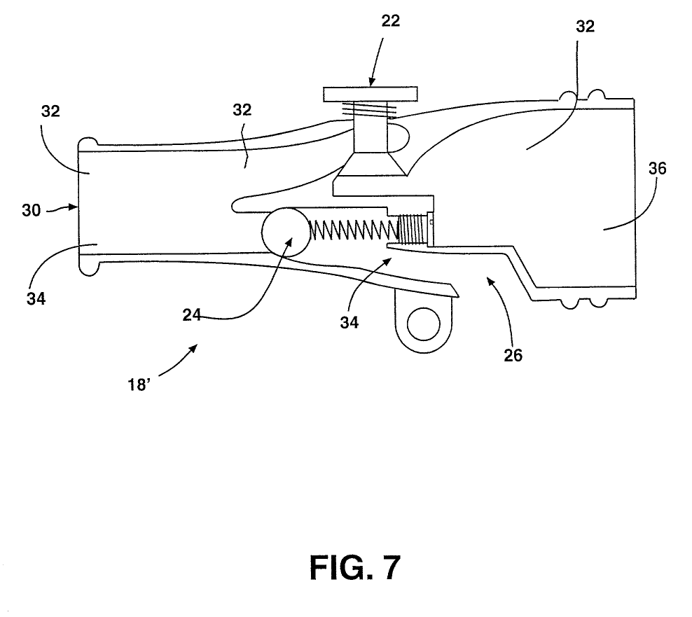

A second embodiment of the mouthpiece 18′ utilized with the present invention can be found in FIGS. 6 and 7. This embodiment further has a vent 26 for allowing the user to exhale out of the device rather than back into the air bladder. Utilizing such a vent 26 would allow a user to exhale into the mouth opening 30, through the inlet channel 34, past the second valve means 24, and out the vent channel 26.

While there is shown and described the present preferred embodiment of the invention, it is to be distinctly understood that this invention is not limited thereto but may be variously embodied to practice within the scope of the following claims.

I claim:

1. A portable emergency breathing apparatus comprising:
an inflatable air bladder, said bladder further comprising at least one handle for allowing a user to grasp said bladder, said bladder further comprising an outlet means for delivering air stored within the bladder to a mouthpiece, said outlet means able to fluidly connect with said mouthpiece;
at least one air inlet means for allowing said air bladder to be inflated with air; and
a mouthpiece for communicating air from said air bladder to a user's mouth, wherein
said mouthpiece comprises at least one valve means for allowing a user selectively release said air from said air bladder through said mouthpiece, said mouthpiece able to fluidly connect with said air bladder through connection with said outlet means.

2. The portable emergency breathing apparatus of claim 1, wherein said inflatable air bladder is made of an elastic material.

3. The portable emergency breathing apparatus of claim 1, wherein said bladder comprises two handles.

4. The portable emergency breathing apparatus of claim 1, wherein said air inlet means attaches to said bladder.

5. The portable emergency breathing apparatus of claim 1, wherein said mouthpiece further comprises a second valve means and a vent channel for allowing the user to open said second valve means and exhale out said vent channel.

6. An emergency breathing apparatus for use with an air storage means, said apparatus comprising:
an apparatus body, having a first end and a second end;
a mouth opening in said first end of said apparatus body for cooperation with a person's mouth, said mouth opening fluidly connected to an outlet channel and an inlet channel;
said outlet channel for supplying a supply of air from an air storage means to a person's mouth, said outlet channel extending through said apparatus body from said mouth opening to an air storage opening;
a first valve means interposed in said outlet channel between said mouth opening and said air storage opening, said first valve means able to move between a first position in which said outlet channel is closed to a second position in which said outlet channel is open, said first valve means further comprising a means for operating said first valve means;
said inlet channel for allowing a person to exhale a supply of air through said apparatus body and into said air storage device, said inlet channel extending through said apparatus body from said mouth opening to said air storage opening;
a second valve means interposed in said inlet channel between said mouth opening and said air storage opening, said second valve means able to move between a first position in which said inlet channel is closed to a second position in which said inlet channel is open, said second valve means further comprising a means for operating said second valve means; and
an air storage opening for fluid cooperation with an air storage means, said air storage opening comprising means for fluidly attaching to said air storage means.

7. The emergency breathing apparatus of claim 6 wherein said first valve means is by default in its first position in which said outlet channel is closed.

8. The emergency breathing apparatus of claim 6 wherein said second valve means is by default in its first position in which said inlet channel is closed.

9. The emergency breathing apparatus of claim 6 wherein said second valve means is a spring loaded check valve able to be opened by applying a positive pressure through the mouth opening and into the inlet channel, said check valve closing when said positive pressure is released.

10. The emergency breathing apparatus of claim 6 wherein said first valve means is able to be manually activated.

11. The emergency breathing apparatus of claim 6 wherein said air storage means is an air bladder.

12. The emergency breathing apparatus of claim 11 wherein said air storage means further comprises at least one handle.

13. The emergency breathing apparatus of claim 11 wherein said air storage means further comprises an air inlet means for allowing a supply of air to be pumped into said air storage means.

14. The emergency breathing apparatus of claim 11 wherein said air storage means further comprises at least one hand strap.

15. The emergency breathing apparatus of claim 11 wherein said air storage means further comprises at least one loop able to hold a flashlight.

16. The emergency breathing apparatus of claim 6, wherein said means for fluidly attaching said apparatus body to said air storage means is at least one rib located on an outside surface of said apparatus body, wherein said air storage means is an air bladder having a neck extending therefrom, said neck able to stretch over said outside surface and held thereon by said rib.

17. An emergency breathing apparatus for use with an air storage means, said apparatus comprising:
an apparatus body, having a first end and a second end;
a mouth opening in said first end of said apparatus body for cooperation with a person's mouth, said mouth opening fluidly connected to an outlet channel and an inlet channel;
said outlet channel for supplying a supply of air from an air storage means to a person's mouth, said outlet channel extending through said apparatus body from said mouth opening to an air storage opening;
a first valve means interposed in said outlet channel between said mouth opening and said air storage opening, said first valve means able to move between a first position in which said outlet channel is closed to a second position in which said outlet channel is open, said first valve means further comprising a means for operating said first valve means;
said inlet channel for allowing a person to exhale a supply of air through said apparatus body and into said air storage device, said inlet channel extending through said apparatus body from said mouth opening to said air storage opening;
a second valve means interposed in said inlet channel between said mouth opening and said air storage opening, said second valve means able to move between a first position in which said inlet channel is closed to a second position in which said inlet channel is open, said second valve means further comprising a means for operating said second valve means; and
an air storage opening in said second end of said apparatus body, said air storage opening for fluid cooperation with an air storage means, said air storage opening comprising means for fluidly attaching to said air storage means.

18. The emergency breathing apparatus of claim 17 wherein said air storage means is an air bladder.

19. The emergency breathing apparatus of claim 17 wherein said air storage means further comprises an air inlet means for allowing a supply of air to be pumped into said air storage means.

20. The emergency breathing apparatus of claim 17, wherein said means for fluidly attaching said apparatus body to said air storage means is at least one rib located on an outside surface of said apparatus body, wherein said air storage means is an air bladder having a neck extending therefrom, said neck able to stretch over said outside surface and held thereon by said rib.
